(19) United States
(12) Patent Application Publication
(10) Pub. No.: US 2006/0106086 A1
(43) Pub. Date: May 18, 2006

(54) Preparation of tegaserod and tegaserod maleate

(76) Inventors: Santiago Ini, Haifa (IL); Anita Liberman, Tel-Aviv (IL); Serguei Fine, Qiriat-Arbaa (IL)

Correspondence Address: KENYON & KENYON LLP, ONE BROADWAY, NEW YORK, NY, 10004, US

(21) Appl. No.: 11/115,871

(22) Filed: Apr. 26, 2005

Related U.S. Application Data
(60) Provisional application No. 60/565,558, filed on Apr. 26, 2004.
(60) Provisional application No. 60/569,045, filed on May 7, 2004.
(60) Provisional application No. 60/662,741, filed on Mar. 17, 2005.

(51) Int. Cl. A61K 31/405; C07D 209/18

(52) U.S. Cl. 514/419; 548/495

(57) ABSTRACT

Provided are processes for preparation of tegaserod and the maleate salt thereof.

### CROSS-REFERENCE TO RELATED APPLICATIONS

This application claims the benefit of U.S. Provisional Application Ser. Nos. 60/565,558 filed Apr. 26, 2004, 60/569,045 filed May 7, 2004, and 60/662,741 filed Mar. 17, 2005, the disclosure of which are incorporated by reference in their entireties herein.

### FIELD OF THE INVENTION

The present invention relates to processes for preparation of tegaserod and salts thereof, particularly tegaserod maleate.

### BACKGROUND OF THE INVENTION

Tegaserod is an aminoguanidine indole 5HT4 agonist for the treatment of irritable bowel syndrome (IBS). Tegaserod maleate has the following chemical name 1-(5-Methoxy-1H-indol-3-ylmethyleneamino)-3-pentylguanidine monomaleate and structure:

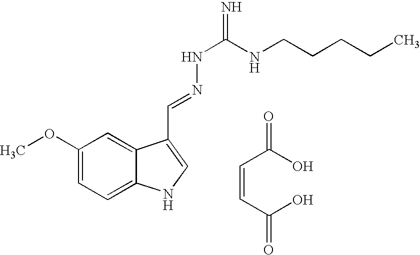

Tegaserod is disclosed in U.S. Pat. No. 5,510,353 A and in its EP equivalent 505322 B1 (example 13 in both of them). Two recent publications after the priority date of the present application, WO2004/085393 and WO2004/014544, provide for crystalline forms of tegaserod maleate, processes for their preparation and their pharmaceutical compositions.

The literature (Buchheit K. H, et al., J. Med. Chem., 1995, 38, 2331) describes a general method for the condensation of aminoguanidines with indole-3-carbaldehydes in methanol in the presence of HCl (pH 3-4). The product obtained after solvent evaporation may be converted to its hydrochloric salt by treatment of the methanolic solution with ether/HCl followed by recrystallization from methanol/diethylether. Tegaserod prepared according to this general method was characterized by a melting point of 155° C. (Table 3 compound 5b).

A recent Chinese patent (CN 1176077) describes a reaction for preparing tegaserod maleate from a mixture of hydroiodic and hydrochloric salts of tegaserod by the addition of maleic acid to the reaction mixture. The reported yield is 69%.

The process for preparing tegaserod maleate disclosed in CN 1176077, U.S. Pat. No. 5,510,353 and Buchheit result in a relatively low yield and/or purity. Addition of maleic acid to a mixture of hydroiodic and/or hydrochloric salt of tegaserod, as suggested in CN 1176077, may result in a mixture of hydrochloric, hydroiodic and maleic acid salt. In addition, reactions under excessive acidic conditions (pH below 3.5) may result in the hydrolysis of the product.

There is a need in the art for additional processes for preparation of tegaserod and its salt suitable for industrial scale.

### SUMMARY OF THE INVENTION

In one aspect the present invention provides a process for preparing tegaserod comprising reacting N-amino-N′-pentylguanidine hydroiodide (AGP-HI) with 5-Methoxy-1H-indole-3-carbaldehyde (5-MICHO) in water under acidic or basic conditions to obtain tegaserod, and recovering the tegaserod. The tegaserod may be converted to the maleate.

In another aspect, the present invention provides a process for preparing tegaserod comprising the steps of:
a) reacting N-amino-N′-pentylguanidine hydroiodide (AGP-HI) and 5-Methoxy-1H-indole-3-carbaldehyde (5-MICHO) under basic or acidic conditions in two phase system of a water immiscible organic solvent and water to obtain tegaserod; and
b) recovering the tegaserod.
   The tegaserod may be converted to the maleate.

In another aspect, the present invention provides a process for preparing tegaserod comprising reacting N-amino-N′-pentylguanidine hydroiodide (AGP-HI) with 5-Methoxy-1H-indole-3-carbaldehyde (MICHO) in an organic solvent under basic conditions to obtain tegaserod and recovering the tegaserod. The tegaserod may be converted to the maleate.

In another aspect the present invention provides a process for preparing tegaserod maleate comprising reacting N-amino-N′-pentylguanidine hydroiodide (AGP-HI) with 5-Methoxy-1H-indole-3-carbaldehyde (5-MICHO) in water or an organic solvent in the presence of maleic acid to precipitate tegaserod maleate, with the proviso that another acid is not used.

In another aspect the present invention provides a process for preparing tegaserod maleate comprising combining a solution of tegaserod acetate in ethyl acetate with a solution of maleic acid in ethyl acetate to obtain a mixture, and recovering the tegaserod maleate.

In another aspect the present invention provides a process for preparing tegaserod maleate comprising combining a mixture of tegaserod hemi-maleate hemihydrate in a C₁-C₈ alcohol acetonitrile, methyl t-butyl ether, C₆ to C₁₂ aromatic solvent ethyl acetate, optionally in mixture with water, with a solution of maleic acid in ethyl acetate having up to 10% water by volume to obtain tegaserod maleate, and recovering the tegaserod maleate.

In another aspect the present invention provides Tegaserod in solid state having a purity of at least about 95% as area percentage HPLC.

In another aspect the present invention provides tegaserod having less than about 1% as area percentage HPLC of an impurity characterized by an HPLC RRT of about 1.06 and a molecular weight of 403.

### DETAILED DESCRIPTION OF THE INVENTION

As used herein, the term “AGP.HI” refers to N-amino-N′-pentylguanidine hydroiodide. As used herein, the term “5-MICHO” refers to 5-Methoxy-1H-indole-3-carbaldehyde. As used herein, the term “TGS” refers to tegaserod. “Tegaserod” as used herein means “tegaserod base” or “tegaserod free base”. As used herein, the term “TEA” refers to triethylamine. As used herein, the term “MA” refers to maleic acid. As used herein, the term “RT” refers to room temperature. As used herein, the term “RM” refers to reaction mixture. As used herein, the term “RRT” refers to relative retention time. As used herein, the term “one pot” means that the reaction is conducted without isolation of tegaserod free base as a solid from the reaction mixture.

The present invention provides a method for the preparation of tegaserod and its maleate salt in water in the presence of base or acid under mild conditions. Reactions in water generally result in a higher yield and purity profile than reaction carried out in organic solvents. Preferably, water free of organic solvent is used.

In addition, the reactions in the presence of bases (organic and inorganic) are extended to organic solvents. Preparation of tegaserod in the presence of a base is suitable for industrial scale, inter alia, since the product of the reaction is tegaserod free base instead of the hydroiodic and hydrochloric salt. An acid intermediate can be avoided altogether.

The use of water as a solvent is suitable for industrial scale due to the resistance of hydrazones to aqueous hydrolysis. The formation of hydrazones is catalyzed by both general acids and general bases. General base catalysis of dehydration of the tetrahedral intermediate involves nitrogen deprotonation concerted with elimination of hydroxide ion as shown in the Scheme (Sayer J. M., et al. J. Am. Chem. Soc. 1973, 95, 4277). 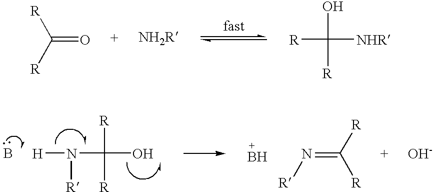
In many cases, the equilibrium constant for their formation in aqueous solution is high. The additional stability may be attributed to the participation of the atom adjacent to the nitrogen in delocalized bonding. 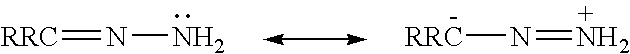
In order to obtain only the maleic salt, the product when using an acid halide (HA) or other acids has to first be converted into the free base, before the addition of maleic acid (Path a), which results in an additional step to the synthesis. On the other hand, the reaction of the present invention in the presence of organic or inorganic base results in the formation of tegaserod free base which gives only the maleate salt after the addition of maleic acid (Path b).

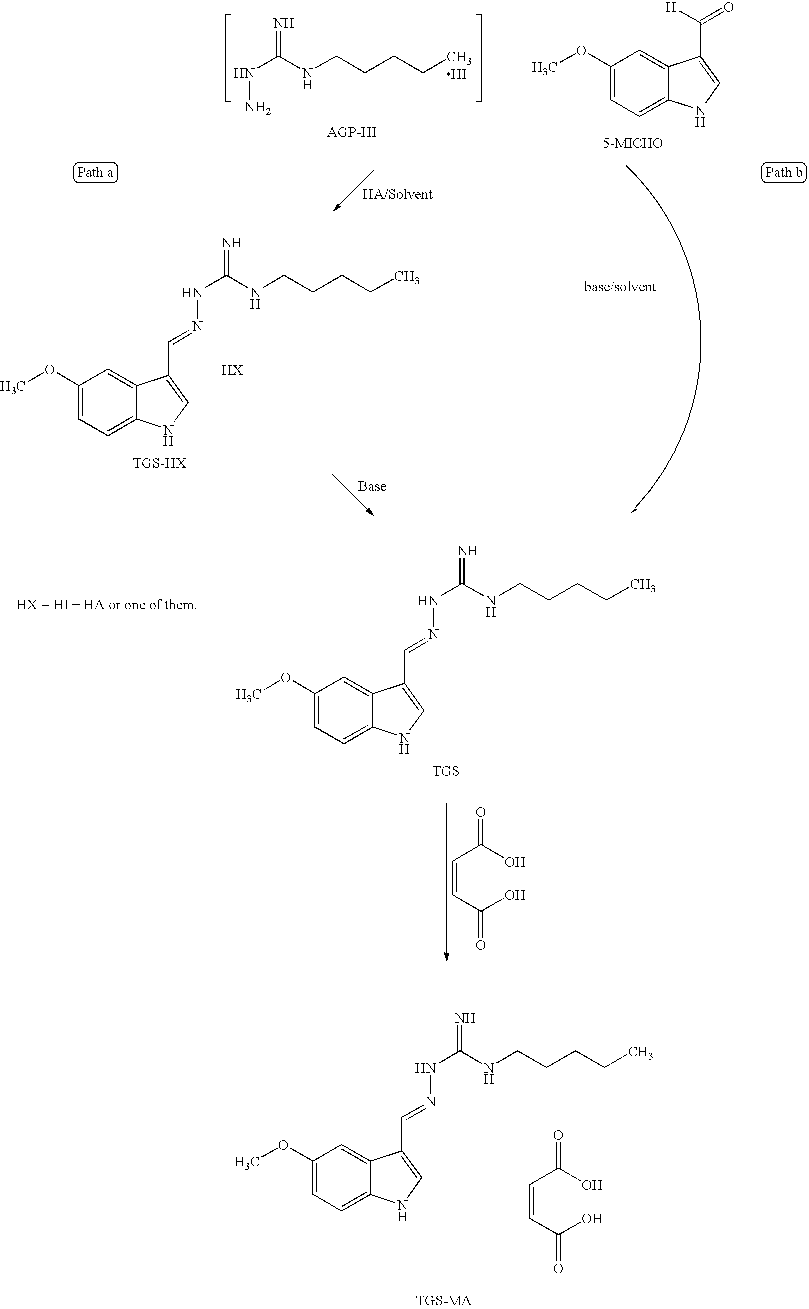

The use of water as a solvent in the synthesis of tegaserod, results in an improvement of the purity of the product as is exemplified in Table 1. Reactions in water in the presence of hydrochloric acid proceed with higher purity rather than reactions performed in methanol under the same conditions (Entry 3 vs. Entry 1 in Table 1). Moreover, reactions performed in water in the presence of organic or inorganic bases, obtain better results than the reactions performed in methanol under similar conditions (Entry 5 and 6 vs. Entry 4 in Table 1). In addition, in all the reactions performed in water the chemical yield are better or similar to the 69% reported for the 3-[[5-(Benzyloxy)-1H-indol-3-yl]methylene]-N-pentylcarbazimidamide Hydrochloride, an analogue of tegaserod hydrochloride salt (Buchheit K. H, et al., J. Med. Chem., 1995, 38, 2331). Preferably, the yield for tegaserod is at least about 85%, more preferably at least about 90% when using water.

TABLE 1
Preparation of tegaserod free base under different conditions.

| | | | | HPLC (%) | | |
|---|---|---|---|---|---|---|
| | | | | | | RRT = 1.06 |
| | | | | | | M.W = 403 |
| Entry | Solvent | Additive | Sample | MICHO | TGS | g/mol |
| 1^{a} | MeOH (RT) | HCl | crude | 1.27 | 77.83 | 6.17 |
| 2^{b} | MeOH (RT) | HCl | RM^{c} | 19.43 | 61.57 | 5.66 |
| | | | crude | 14.07 | 67.50 | 5.30 |
| 3 | H₂O | HCl | crude | 0.25 | 87.42 | 9.90 |
| | (Reflux) | | | | | |
| 4 | MeOH (RT) | TEA | RM | 0.94 | 83.87 | 10.81 |
| | | | crude | 0.05 | 86.90 | 8.42 |
| | H₂O (RT) | TEA | RM | 1.02 | 91.87 | 4.09 |
| 5 | | | Crude | 1.93 | 94.02 | 2.78 |
| | | | (MA) | | | |
| 6 | H₂O | NaHCO₃ | RM | 0.15 | 91.98 | 3.45 |
| | (Reflux) | | | | | |
| | | | crude | 0.18 | 91.55 | 3.03 |
| 7 | H₂O (RT) | NaOH | crude | 0.26 | 98.80 | 0.20 |

^{a}According to patent U.S. Pat. No. 5,510,353.
^{b}According to Buchheit K. H, et al., J. Med. Chem., 1995, 38, 2331)
^{c}Reaction mixture

According to one embodiment, the present invention provides a process for preparing tegaserod base comprising reacting AGP-HI with 5-MICHO in water under basic or acidic conditions to form tegaserod.

When the reaction is carried out under basic conditions with water, an organic or inorganic base may be used. The organic base is preferably a C₃ to C₈ alkyl amine such as trialkylamine (preferably triethylamine), and pyridine. The inorganic base may be an alkali/alkaline earth-hydroxide or carbonate, preferably K₂CO₃, Na₂CO₃, NaHCO₃, NaOH, KOH, more preferably NaOH. The reaction is preferably carried out at a pH range of 7 to 14, more preferably of about 9 to 14. The temperature range during the reaction is preferably of about 5° C. to reflux temperature. When a tertiary amine is used, the tertiary amine may also act as a solvent, thus, the reaction may be carried out in the presence of the tertiary amine in neat form, i.e. without the use of an additional solvent.

When the reaction is carried out under acidic conditions with water, an organic or inorganic acid may be used. An organic acid such as p-toluensulfonic acid, pyridinium p-toluenesulfonic acid, methanesulfonic acid, acetic acid or maleic acid may be used. In another embodiment, an inorganic acid such as HCl, HBr, H₃PO₄ or H₂SO₄ may be used. The pH range during the reaction is preferably of 1 to 7, more preferably of about 3 to 4. The temperature range during the reaction is preferably of about 5° C. to about reflux temperature of water Before conversion to the maleate salt, a base may be used to neutralize the acid used in the process or to eliminate undesirable salts.

The present invention also provides for preparing tegaserod by reacting AGP-HI with 5-MICHO under basic conditions in an organic solvent. Reactions in organic solvent under basic conditions generally result in a higher yield and purity profile than reactions carried out under acidic conditions. In addition, the tegaserod tends to decompose under acidic conditions with a pH of less than 3. The organic solvent may be a nitrile, a tertiary amine, C₁ to C₈ alcohol such as methanol (MeOH) or iso propyl alcohol (IPA), or acetonitrile, or a C₂ to C₈ ether such as methyl tertbutyl ether or diisopropyl ether, or a C₃ to C₈ ester such as ethyl acetate. It is also possible to carry out the reaction in the tertiary amine without the use of water or an organic solvent. The reaction is carried out at a pH range of 7 to 14, more preferably about 9 to 14. The temperature range during the reaction is of about 5° C. to the reflux temperature of the selected solvent. The organic base is preferably a C₃ to C₈ alkyl amine such as trialkylamines (preferably triethylamine), and pyridine. The inorganic base may be an alkali/alkaline earth-hydroxide or carbonate, preferably K₂CO₃, Na₂CO₃, NaHCO₃, NaOH, KOH, more preferably NaOH.

According to the another embodiment, the present invention provides a process for preparing tegaserod base by reacting AGP-HI with 5-MICHO under basic conditions in a two phase system. A preferred solvent mixture is that of water and a C₆ to C₁₂ aromatic hydrocarbon such as xylene, propylbenzene, benzene and toluene. An organic or inorganic base may be used. The organic base is preferably a C₃ to C₈ alkyl amine such as trialkylamines (preferably triethylamine), and pyridine. The inorganic base may be an alkali/alkaline earth-hydroxide or carbonate, preferably K₂CO₃, Na₂CO₃, NaHCO₃, NaOH, KOH, more preferably NaOH. In one embodiment, AGP-HI is dissolved in water to form a solution. The aqueous solution is in contact with a water immiscible solvent, and they together form a two phase system. Subsequently, 5-MICHO and a base are added to the two phase system. The resulting tegaserod is recovered by conventional techniques such as filtration from the reaction mixture. The two phase system results in a product with higher purity as illustrated in Example 10. The temperature range during the reaction is preferably of about 5° C. to about reflux temperature.

The tegaserod may be recovered in various manners. When using water as a solvent, the tegaserod may be recovered by moving the tegaserod into an organic solvent by extraction, followed by removal of the organic solvent, such as by evaporation under ambient or reduced pressure (Pressure of below 1 atmosphere, more preferably below about 100 mmHg). When using an acid, the mixture is preferably neutralized before the extraction with an organic solvent. The organic solvent is preferably ethyl acetate or dichloromethane, more preferably ethyl acetate. The organic solvent may be washed with water before recovery to remove water soluble impurities. A preferred pH range for extraction is of 7 to 14, preferably of about 9 to 14. The tegaserod base may also be precipitated out of water or an organic solvent.

The tegaserod base may be converted to the maleate salt after recovery. In a preferred embodiment, tegaserod recovered from the organic solvent after extraction or precipitated out of H₂O, is combined with maleic acid to provide tegaserod maleate, optionally tegaserod maleate Form A. Tegaserod maleate Form A is characterized by an X-ray Diffraction pattern having peaks at 5.4, 5.9, 6.4, 10.8, 11.5, 12.0, 14.8, 15.4, 16.2, 18.1, 19.4, 21.7, 23.9, 26.8, 29.7, +/−0.2 degrees two theta. Tegaserod maleate Form A is disclosed in U.S. Appl. No. 60/530,278, filed on Dec. 16, 2003, incorporated herein by reference.

It is also possible to convert tegaserod base to the maleate without recovering the base. For example, tegaserod maleate may be prepared by adding maleic acid to a solution of tegaserod base in an organic solvent, and recovering the crude tegaserod maleate. The organic solvent may be methanol, ethanol, iso-propanol, n-propanol, acetonitrile, n-butanol, acetone, dioxane, methyl ethyl ketone, tetrahydrofuran, ethyl lactate, ethyl acetate or dimethyl carbonate. The evaporation of the solvent is preferably carried out under reduced pressure, more preferably at a pressure below about 100 mmHg.

The tegaserod base may also be converted to tegaserod maleate by adding maleic acid to a solution of tegaserod base in water, an organic solvent or mixtures thereof, with a mixture of water and one of acetone, methanol and ethyl acetate being preferred. The crystals may be recovered by conventional techniques such as filtration.

The recovery of tegaserod is particularly convenient when using maleic acid both as a catalyst and a source of maleic acid. In this process, tegaserod maleate is prepared by reacting AGP-HI with 5-MICHO in water or an organic solvent under acidic conditions created by use of maleic acid. Use of another acid such as hydrogen halide is not necessary. After formation of tegaserod, tegaserod maleate precipitates out of the solution. The organic solvent is preferably a C₁-C₈ alcohols, acetonitrile, methyl t-butyl ether, toluene (either alone or mixed with water), ethyl acetate and iso propyl alcohol (IPA).

In a one pot embodiment of the present invention, AGP-HI and 5-MICHO are reacted in water under acidic or basic conditions, preferably basic conditions. Under basic conditions, the reaction may be carried out at room temperature without heating. After completion of the reaction, maleic acid is added to precipitate the maleate salt, without recovery of tegaserod from the reaction mixture with the methods described above. Alternatively, a water immiscible solvent may be added after the reaction to move the tegaserod to the organic solvent under suitable pH, such as ethyl acetate, followed by addition of maleic acid to precipitate the maleate from the organic solvent without isolation of tegaserod. It is also possible to carry out the reaction in an organic solvent under basic conditions.

The maleic acid in the processes of the present invention is preferably added as a solution of the same solvent that contains tegaserod base.

Tegaserod maleate may also be prepared from tegaserod acetate by adding maleic acid. In one embodiment, a solution of tegaserod acetate is heated in ethyl acetate preferably at a temperature of about RT to about 80° C., more preferably about 65° C. The solution is then combined with a solution of maleic acid in ethyl acetate, preferably containing up to about 10% water by volume. The resulting mixture is then stirred and the tegaserod maleate recovered preferably by filtration. The tegaserod maleate is then preferably dried at a temperature of about 30° C. to about 45° C., more preferably under a pressure of less than about 100 mmHg.

Tegaserod maleate may also be prepared from tegaserod hemi-maleate hemihydrate. In one embodiment, a solution of maleic acid in ethyl acetate containing up to about 10% water by volume is added to a mixture of tegaserod hemi-maleate hemihydrate and ethyl acetate. Preferably, the temperature is about room temperature. The resulting mixture is then stirred and the tegaserod maleate recovered, preferably by filtration. The tegaserod maleate is then preferably dried at a temperature of about 30° C. to about 45° C., more preferably under a pressure of less than about 100 mm Hg. Other suitable organic solvents include a C₁-C₈ alcohol (such methanol, ethanol, propanol), acetonitrile, methyl t-butyl ether, C₆ to C₁₂ aromatic solvent (such xylene, toluene, benzene and propyl-benzene), ethyl acetate, optionally in a mixture with water. Te organic solvent may be used to dissolve the maleic acid as well.

The tegaserod base obtained with the process of the present invention is substantially pure. Preferably, the tegaserod base has an impurity of at least about 95%, more preferably at least about 98%, and most preferably at least about 99% as area percentage HPLC carried out according to the disclosure of the present invention. The tegaserod base of the present invention is also substantially free of an impurity characterized by an RRT of 1.06 and a molecular weight of 403, preferably containing less than about 1.00, more preferably less than about 0.50 and most preferably about 0.20 of the impurity as area percentage HPLC.

Pharmaceutical formulations of the present invention contain tegaserod maleate as prepared by the processes of the present invention. The pharmaceutical composition may contain only a single form of tegaserod base or maleate, or a mixture of various forms of tegaserod maleate, with or without amorphous form. In addition to the active ingredient(s), the pharmaceutical compositions of the present invention may contain one or more excipients or adjuvants. Selection of excipients and the amounts to use may be readily determined by the formulation scientist based upon experience and consideration of standard procedures and reference works in the field.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dixoide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate. Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions of the present invention, the active ingredient and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the present invention, a liquid composition may also contain a buffer such as guconic acid, lactic acid, citric acid or acetic acid, sodium guconate, sodium lactate, sodium citrate or sodium acetate.

Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and losenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

The dosage used is preferably from about 1 mg to about 10 mg of tegasorad base equivalent, more preferably from about 2 to about 6 mg. The pharmaceutical compositions of the present invention, used to treat irritable bowel syndrome in a mammal such as a human, are preferably in the form of a coated tablet, and are administered on an empty stomach twice a day, for a period of about 4 to about 6 weeks. Additional administration may occur if the patient responds positively to the treatment. Generally, each 1.385 mg of tegaserod as the maleate is equivalent to 1 mg of tegaserod free base. A possible formulation is as follows: crospovidone, glyceryl monostearate, hydroxypropyl methylcellulose, lactose monohydrate, poloxamer 188, and polyethylene glycol 4000.

### EXAMPLES

### HPLC Method for Detecting the Level of the Impurities

Column: Atlantis dcl8(150*4.6),
Mobile phase: A.80% KH₂PO₄(0.02M) pH=5, 20% acetonitrile(ACN), B.100% ACN.
Gradient: time 0=A: 100 B: 0, time 25 min=A:50%, B:50%, time 30 min=A:50%, B:50%, + 10 minutes of equilibration time.
Wavelength=225 nm
Sample concentration: 0.5 mg/mL
Temperature=25° C.

### Example 1

Preparation of Tegaserod maleate in water with HCl.

To a mixture of AGP.HI (10.88 g, 0.04 mol) in 25 mL water was added 5-MICHO (3.50 g, 0.02 mol) followed by HCl (37%) until pH 4. The mixture was heated to reflux for 1 hour and then cooled to room temperature. To the resulting slurry was added a solution of NaHCO₃ (10%) until pH 9, and heated to 65° C. for 20 minutes. After cooling, 100 mL of EtOAc were added, and the organic phase washed with water. A solution of maleic acid (3.48 g, 0.03 mol) in 100 mL EtOAc was added, and the resulting solid was filtered off and washed with EtOAc to give 6.27 g of crude tegaserod maleate with a purity of 99.70% (by HPLC).

### Example 2

Preparation of Tegaserod maleate in water with HCl in two steps.

a. Preparation of Tegaserod free base.

To a mixture of AGP.HI (163.3 g, 0.6 mol) in 375 mL water was added 5-MICHO (52.5 g, 0.3 mol) followed by HCl (37%) until pH 4. The mixture was heated to reflux for 1 hour and then cooled to room temperature. To the resulting slurry was added a liter of a solution of NaHCO₃ (10%) until pH 9, and heated to 65° C. for one hour. After cooling, 1500 mL of EtOAc were added, and the organic phase washed with water. The remaining organic phase was evaporated to dryness to give tegaserod free base with a purity of 87.42% (by HPLC).

b. Preparation of Tegaserod maleate.

To a solution of 2 g of tegaserod free base in MeOH was added a solution of maleic acid (1.28 g, 0.011 mol) in 10 mL MeOH. The resulting solid was filtered off and washed with MeOH to give 1.09 g of crude tegaserod maleate with a purity of 96.81% (by HPLC).

### Example 3

Preparation of Tegaserod maleate in water with TEA.

To a mixture of AGP.HI (10.88 g, 0.04 mol) in 100 mL water was added 5-MICHO (3.50 g, 0.02 mol) followed by TEA (11.0 mL, 0.08 mol) and stirred at room temperature. After one hour, 25 mL of EtOAc was added, and the organic phase washed with water. A solution of maleic acid (3.48 g, 0.03 mol) in 100 mL EtOAc was added, and the resulting solid was filtered off and washed with EtOAc to give 7.92 g of crude tegaserod maleate with a purity of 94% (by HPLC).

### Example 4

Preparation of Tegaserod maleate in water with NaHCO₃.

To a mixture of AGP.HI (10.88 g, 0.04 mol) in 100 mL water was added 5-MICHO (3.50 g, 0.02 mol) followed by NaHCO₃ (6.72 g, 0.08 mol) and heated to reflux for 1 hour. After cooling, 50 mL of EtOAc was added, and the organic phase washed with water. A solution of maleic acid (3.48 g, 0.03 mol) in 100 mL EtOAc was added, and the resulting solid was filtered off and washed with EtOAc to give 6.71 g of crude tegaserod maleate with a purity of 98% (by HPLC).

### Example 5

Preparation of Tegaserod maleate in water with NaHCO₃ in two steps.

a. Preparation of Tegaserod free base.

To a mixture of AGP.HI (32.66 g, 0.12 mol) in 300 mL water was added 5-MICHO (10.51 g, 0.06 mol) followed by NaHCO₃ (20.16 g, 0.24 mol) and heated to reflux for 1 hour. After cooling, 150 mL of EtOAc was added, and the organic phase washed with water and evaporated to dryness to give 20.4 g of tegaserod free base (91.55% purity by HPLC).

b. Preparation of Tegaserod maleate.

To a solution of 2 g of the resulting tegaserod free base in 8 mL MeOH was added a solution of maleic acid (1.28 g, 0.011 mol) in 5 mL MeOH. The resulting solid was filtered off and washed with MeOH to give 2.1 g of crude tegaserod maleate with a purity of 99.63% (by HPLC).

### Example 6

Preparation of Tegaserod maleate in water with Na₂CO₃.

To a mixture of AGP.HI (10.88 g, 0.04 mol) in 100 mL water was added 5-MICHO (3.50 g, 0.02 mol) followed by Na₂CO₃ (4.24 g, 0.04 mol) and heated to reflux for 1 hour. After cooling, 50 mL of EtOAc was added, and the organic phase washed with water. A solution of maleic acid (3.48 g, 0.03 mol) in 100 mL EtOAc was added, and the resulting solid was filtered off and washed with EtOAc to give 6.48 g of crude tegaserod maleate with a purity of 98.2% (by HPLC).

### Example 7

Preparation of Tegaserod maleate in MeOH with TEA in two steps.

a. Preparation of Tegaserod Free Base

To a mixture of AGP.HI (10.88 g, 0.04 mol) in 25 mL MeOH was added 5-MICHO (3.50 g, 0.02 mol) followed by triethylamine (11.0 mL, 0.08 mol). After 1 h at room temperature the mixture was evaporated to dryness, and washed with water, giving 5.79 g of tegaserod free base (86.90% purity by HPLC).

b. Preparation of Tegaserod Maleate

To a solution of 2 g of the resulting tegaserod free base in 10 mL MeOH was added a solution of maleic acid (1.16 g, 0.01 mol) in water. The resulting solid was filtrated and washed with water to give 1.45 g of crude tegaserod maleate as a white solid (94.60% purity by HPLC). Crystallization in MeOH improved the purity to 98.94% by HPLC.

### Example 8

Preparation of Tegaserod maleate in IPA with K₂CO₃.

To a mixture of AGP.HI (10.88 g, 0.04 mol) in 25 mL IPA was added 5-MICHO (3.50 g, 0.02 mol) followed by K₂CO₃ (5.53 g, 0.04 mol). After 22 h at room temperature the mixture was washed with brine. The organic phase was treated with a solution of maleic acid (3.48 g, 0.03 mol) in IPA. The resulting solid was filtrated and washed with IPA to give 3.26 g of a white solid (98.97% purity by HPLC).

### Example 9

Preparation of Tegaserod maleate in TEA.

To a mixture of AGP.HI (10.88 g, 0.04 mol) and 5-MICHO (3.50 g, 0.02 mol) was added 11 mL of TEA (0.08 mol). After 2 h at room temperature 25 mL of EtOAc were added and the mixture was stirred for 1 h. The resulting solid was filtrated and washed with 25 mL EtOAc, to give 5.7 g of crude.

2 g of the residue was dissolved in 13 mL MeOH and treated with 7 mL of a solution of maleic acid (2.7 g, 0.023 mol) in water. The resulting solid was filtered and washed with water to give 1.5 g of tegaserod maleate (99.26% purity by HPLC). Crystallization of the solid in MeOH improved the purity to 99.89% by HPLC.

### Example 10

Preparation of Tegaserod maleate in toluene/water with NaHCO₃.

a. Preparation of Tegaserod Free Base

To a mixture of AGP.HI (10.88 g, 0.04 mol) in 200 mL of water/toluene 1:1 was added 5-MICHO (3.50 g, 0.02 mol) followed by NaHCO₃ (6.72 g, 0.08 mol) and heated to reflux for 1 hour. After cooling, the solid was filtrated out of the mixture and washed with water. After drying 6.25 g of tegaserod free base was obtained (93.8% purity by HPLC).

b. Preparation of Tegaserod Maleate

To a solution of 3 g of the product in 10 mL MeOH was added a solution of maleic acid (2.31 g, 0.02 mol) in 10 mL water. The resulting solid was filtered off and washed with a solution of MeOH/water to give 2.50 g of crude tegaserod maleate with a purity of 96.6% (by HPLC).

### Example 11

Preparation of Tegaserod maleate in water with NaOH.

a. Preparation of Tegaserod Free Base

To a mixture of AGP.HI (10.88 g, 0.04 mol) in 25 mL of water was added 5-MICHO (3.50 g, 0.02 mol) followed by NaOH (2 g, 0.05 mol) and stirred at room temperature. After 3 hours 50 mL of EtOAc was added, and the organic phase washed with water and evaporated to dryness to give 5.6 g of tegaserod free base (98.80% purity by HPLC).

b. Preparation of Tegaserod maleate.

To a solution of 1.6 g of tegaserod free base in 15 mL ethyl acetate was added a solution of maleic acid (0.7 g, 0.006 mol) in 5 mL ethyl acetate. The resulting solid was filtered off and washed with ethyl acetate to give 1.65 g of crude tegaserod maleate, with a purity of 99.87% (by HPLC)

### Example 12

Preparation of Tegaserod maleate in water with maleic acid.

To a mixture of AGP.HI (10.88 g, 0.04 mol) in 25 mL of water was added 5-MICHO (3.50 g, 0.02 mol) followed by maleic acid (9.3 g, 0.08 mol) and heated to reflux for 1 hour. After cooling, the solid was filtrated out of the mixture and washed with water. After drying 6.92 g of tegaserod maleate crude was obtained (92.4% purity by HPLC).

### Example 13

Preparation of Tegaserod maleate in methanol with maleic acid.

To a mixture of AGP.HI (10.88 g, 0.04 mol) in 25 mL of methanol was added 5-MICHO (3.50 g, 0.02 mol) followed by maleic acid (9.29 g, 0.08 mol) and heated to reflux for 2 hours. After cooling, the solid was filtrated out of the mixture and washed with water. After drying 6.51 g of tegaserod maleate crude was obtained (97.4% purity by HPLC).

### Example 14

Preparation of Tegaserod maleate in water with NaOH in one pot.

To a mixture of AGP.HI (10.88 g, 0.04 mol) in 25 mL of water was added 5-MICHO (3.50 g, 0.02 mol) followed by NaOH (2 g, 0.05 mol) and stirred at room temperature. After 4 hours a solution of maleic acid (4.35 g, 0.0375 mol) in 25 mL water was added, and the reaction mixture was stirred overnight. The resulting solid was filtered off and washed with water to give 7.87 g of crude tegaserod maleate (99.16% purity by HPLC).

### Example 15

Preparation of Tegaserod maleate in water with NaOH in one pot.

To a mixture of AGP.HI (174.2 g, 0.64 mol) in 362 mL of water was added 5-MICHO (56.2 g, 0.32 mol) followed by NaOH (68.1 g, 47%) and stirred at room temperature. After 4.5 hours, 640 mL of EtOAc was added, and the organic phase washed with water, treated with active carbon and filtrated through hyper flow bed. A solution of maleic acid (44.57 g, 0.38 mol) in 415 mL ethyl acetate/water 97:3 was added, and the reaction mixture was heating to 65° C. and stirrer overnight. The resulting solid was filtered off and washed with water and ethyl acetate to give 121.4 g of crude tegaserod maleate (up to 99.88% purity by HPLC).

### Example 16

Preparation of Tegaserod maleate (from Tegaserod acetate).

To a solution of 8.2 g of tegaserod acetate in 15 mL ethyl acetate heated to 65° C. was added a solution of 3.3 g maleic acid in 5 ml ethyl acetate/water 95:5, and the mixture was stirred at the same temperature for an additional 2 hours, followed by cooling to room temperature and stirring overnight. The resulting solid was filtered off and washed with ethyl acetate/water 95:5. After drying on vacuum oven at 45° C. for 15 hours, 9.18 g of tegaserod maleate were obtained. Tegaserod acetate is prepared according to Examples 19, 20 and 21 of U.S. application Ser. No. 11/015,875 and PCT/US04/42822.

Example 19 of U.S. application Ser. No. 11/015,875 reads as follows:

A slurry of tegaserod base amorphous (6 g) in 50 mL ethyl acetate was stirred at 20-30° C. for 24 hours. The solid was filtrated and washed with 15 mL of same solvent and dried in a vacuum oven at 40° C. for 16 hours.

Example 20 of U.S. application Ser. No. 11/015,875 reads as follows:

A slurry of tegaserod base amorphous (6 g) in 50 mL ethyl acetate was stirred at reflux for 24 hours. The solid was filtrated and washed with 15 mL of same solvent and dried in a vacuum oven at 40° C. for 16 hours.

Example 21 of U.S. application Ser. No. 11/015,875 reads as follows:

To a slurry of tegaserod maleate Form A (15 g) in EtOAc (210 mL) and water (210 mL) was added 38.4 g of NaOH 47%. The mixture was stirred overnight and the resulting white solid was isolated by filtration and washed with 100 mL of water. Drying in vacuum oven at 40° C. for 16 hours gives 12.38 g (90% yield). Tegaserod acetate was characterized by ¹H and ¹³C—NMR.

### Example 17

General method for the preparation of Tegaserod maleate Form A from crystallization.

Tegaserod maleate (1 g) was combined with the appropriate solvent (5 mL), and heated to reflux. Then, additional solvent was added until complete dissolution. After the compound was dissolved, the oil bath was removed and the solution was cooled to room temperature. The solid was filtrated and washed with 5 mL of the same solvent and dried in a vacuum oven at 40° C. for 16 hours.

| | Total | Form before | Form After |
|---|---|---|---|
| Solvent | Volume (mL) | Drying | Drying |
| Acetonitrile | 80 | A | A |
| Butyl lactate | 10 | A | A |
| Methyl ethyl ketone | 60 | A | A |
| sec-butanol | 40 | A | A |
| Dioxane | 120 | A | A |
| Methanol/water 20:80 | 60 | A | A |
| Ethanol/water 20:80 | 60 | A | A |
| Isopropanol/water 1:1 | 7 | A | A |
| Isopropanol/water | 43 | A | A |
| 20:80 | | | |
| Acetonitrile/water 1:1 | 7 | A | A |
| Acetonitrile/water | 47 | A | A |
| 20:80 | | | |
| Chloroform/2- | 7 | A | A |
| ethoxyethanol 1:1 | | | |
| Chloroform/2- | 13 | A | A |
| ethoxyethanol 25:75 | | | |
| Water/2- | 5 | A | A |
| ethoxyethanol 1:1 | | | |
| n-BuOH | 6 | A | A |
| Water/1-methyl-2- | 8 | D | A |
| pyrrolidone 75:25 | | | |

### Example 18

Preparation of Tegaserod maleate in water with p-TSOH.

To a mixture of AGP.HI (10.88 g, 0.04 mol) in 25 mL water was added 5-MICHO (3.50 g, 0.02 mol) followed by para-toluenesulfonic acid monohydrate (0.45 g, 0.0024 mol). The mixture was heated to reflux for 4 hour and then cooled to room temperature. The resulting solid was filtered off and washed with water to give 8.32 g of a white solid (84.74% purity by HPLC).

### Example 19

Preparation of Tegaserod Maleate from Tegaserod Hemi-Maleate Hemihydrate

To a solution of 1.72 g of Tegaserod Hemi-maleate hemihydrate in 20 mL ethyl acetate at room temperature was added a solution of 0.134 g maleic acid in 5 ml ethyl acetate/water 95:5, and the mixture was stirred at the same temperature for overnight. The resulting solid was filtered off and washed with ethyl acetate/water 95:5. After drying on vacuum oven at 45° C. for 15 hours, 1.68 g of tegaserod maleate were obtained. Tegaserod Hemi-maleate hemihydrate was prepared according to Example 23 of U.S. application Ser. No. 11/015,875 and PCT/US04/42822.

Example 23 of U.S. application Ser. No. 11/015,875 and PCT/US04/42822 reads as follows:

A solution of maleic acid (2.32 g in 22 mL ethyl acetate/water 97:3) was added to a mixture of tegaserod base in ethyl acetate, and the reaction mixture was heated to 65° C. and stirrer overnight. The resulting solid was filtered off and washed with water and ethyl acetate. Drying in vacuum oven at 40° C. for 16 hours gives 12.19 g of Tegaserod hemi-maleate hemihydrate. Depending on the base polymorph used a solution or slurry is obtained. When using amorphous tegaserod base, a solution is obtained, while when using any other base polymorph of tegaserod, a slurry is obtained.

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way. The examples do not include detailed descriptions of conventional methods. Such methods are well known to those of ordinary skill in the art and are described in numerous publications. All references mentioned herein are incorporated in their entirety.

What is claimed is:

1. A process for preparing tegaserod comprising reacting N-amino-N′-pentylguanidine hydroiodide (AGP-HI) with 5-Methoxy-1H-indole-3-carbaldehyde (5-MICHO) in water under acidic or basic conditions to obtain tegaserod, and recovering the tegaserod.

2. The process of claim 1, wherein the reaction is carried out under basic conditions.

3. The process of claim 2, wherein the base is an inorganic base selected from the group consisting of alkali/alkaline-earth-metal hydroxides and carbonates.

4. The process of claim 3, wherein the base is selected from the group consisting of K₂CO₃, Na₂CO₃, NaOH, KOH and NaHCO₃.

5. The process of claim 2, wherein the base is an organic base selected from the group consisting of C₃ to C₈ tertiary amines.

6. The process of claim 5, wherein the C₃ to C₈ alkyl amine is trialkylamine or pyridine.

7. The process of claim 6, wherein the base is triethylamine.

8. The process of claim 7, wherein the reaction is carried out at a pH range of about 9 to 14.

9. The process of claim 1, wherein the reaction is carried under acidic conditions.

10. The process of claim 9, further comprising neutralizing the acid.

11. The process of claim 9, wherein the acid is an inorganic acid selected from the group consisting of HCl, HBr, H₃PO₄ and H₂SO₄.

12. The process of claim 9, wherein the acid is an organic acid selected from the group consisting of p-toluensulfonic acid, pyridinium p-toluenesulfonic acid, methanesulfonic acid, acetic acid and maleic acid.

13. The process of claim 12, wherein the reaction is carried out at a pH range of about 3 to 4

14. A process for preparing tegaserod maleate comprising preparing tegaserod base of claim 1 and converting it to tegaserod maleate

15. The process of claim 14, wherein converting comprises:
a) suspending or dissolving the tegaserod in an organic solvent;
a) combining the solution or suspension with maleic acid; and
b) recovering the tegaserod maleate as a precipitate or a residue.

16. The process of claim 15, wherein the organic solvent is selected from the group consisting of: methanol, ethanol, iso-propanol, n-propanol, acetonitrile, n-butanol, acetone, dioxane, methyl ethyl ketone, tetrahydrofuran, ethyl lactate, ethyl acetate and dimethyl carbonate.

17. The process of claim 14, wherein converting comprises:
a) preparing a mixture of tegaserod in water;
b) combining the mixture with maleic acid; and
c) recovering the tegaserod maleate as a precipitate.

18. A process for preparing tegaserod comprising the steps of:
c) reacting N-amino-N′-pentylguanidine hydroiodide (AGP-HI) and 5-Methoxy-1H-indole-3-carbaldehyde (5-MICHO) under basic or acidic conditions in two phase system of a water immiscible organic solvent and water to obtain tegaserod; and
d) recovering the tegaserod.

19. The process of claim 18, wherein the water immiscible organic solvent is selected from the group consisting of C₆ to C₁₂ aliphatic or aromatic hydrocarbon.

20. The process of claim 19, wherein the aromatic hydrocarbon is selected from the group consisting of xylene, toluene, benzene and propyl-benzene.

21. A process for preparing tegaserod maleate comprising preparing tegaserod according to claim 18 and converting it to tegaserod maleate.

22. The process of claim 21, wherein converting comprises:
a) suspending or dissolving the tegaserod in an organic solvent;
b) combining the solution or suspension with maleic acid; and
c) recovering the tegaserod maleate as a precipitate or a residue.

23. The process of claim 22, wherein the organic solvent is selected from the group consisting of: methanol, ethanol, iso-propanol, n-propanol, acetonitrile, n-butanol, acetone, dioxane, methyl ethyl ketone, tetrahydrofuran, ethyl lactate, ethyl acetate and dimethyl carbonate.

24. The process of claim 21, wherein converting comprises:
a) preparing a mixture of tegaserod in water;
b) combining the mixture with maleic acid; and
c) recovering the tegaserod maleate as a precipitate.

25. A process for preparing tegaserod comprising reacting N-amino-N′-pentylguanidine hydroiodide (AGP-HI) with 5-Methoxy-1H-indole-3-carbaldehyde (MICHO) in an organic solvent under basic conditions to obtain tegaserod and recovering the tegaserod.

26. The process of claim 25, wherein the organic solvent is selected from the group consisting of: C₁-C₈ alcohols, nitriles, C₂-C₈ ethers, C₃-C₈ esters and tertiary amines.

27. The process of claim 26, wherein the organic solvent is an organic base.

28. The process of claim 27, wherein the organic base is a tertiary amine.

29. The process of claim 28, wherein the organic solvent is selected from the group consisting of: methanol, isopropyl alcohol (IPA), acetonitrile, methyl tert butyl ether and ethyl acetate

30. A process for preparing tegaserod maleate comprising preparing tegaserod according to claim 25 and converting it to tegaserod maleate.

31. The process of claim 30, wherein converting comprises:
a) suspending or dissolving the tegaserod in an organic solvent;
b) combining the solution or suspension with maleic acid; and
c) recovering the tegaserod maleate as a precipitate or residue.

32. The process of claim 31, wherein the organic solvent is selected from the group consisting of: methanol, ethanol, iso-propanol, n-propanol, acetonitrile, n-butanol, acetone, dioxane, methyl ethyl ketone, tetrahydrofuran, ethyl lactate, ethyl acetate and dimethyl carbonate.

33. The process of claim 30, wherein converting comprises:
a) preparing a mixture of tegaserod in water;
b) combining the mixture with maleic acid; and
c) recovering the tegaserod maleate as a precipitate.

34. A process for preparing tegaserod maleate comprising reacting N-amino-N′-pentylguanidine hydroiodide (AGP-HI) with 5-Methoxy-1H-indole-3-carbaldehyde (5-MICHO) in water or an organic solvent in the presence of maleic acid to precipitate tegaserod maleate, with the proviso that another acid is not used.

35. The process of claim 34, wherein the reaction is carried out in an organic solvent.

36. The process of claim 35, wherein the organic solvent is selected from the group consisting of: C₁-C₈ alcohols, acetonitrile, methyl t-butyl ether, and toluene/water.

37. The process of claim 34, wherein the reaction is carried out in water in the absence of an organic solvent.

38. A process for preparing tegaserod maleate comprising combining a solution of tegaserod acetate in ethyl acetate with a solution of maleic acid in ethyl acetate to obtain a mixture, and recovering the tegaserod maleate.

39. The process of claim 38, the heated solution has a temperature of about 40° C. to about 80° C.

40. The process of claim 38, wherein the maleic acid solution contains up to about 10% water by volume.

41. A process for preparing tegaserod maleate comprising combining a mixture of tegaserod hemi-maleate hemihydrate in a C₁-C₈ alcohol acetonitrile, methyl t-butyl ether, C₆ to C₁₂ aromatic solvent ethyl acetate, optionally in mixture with water, with a solution of maleic acid in ethyl acetate having up to 10% water by volume to obtain tegaserod maleate, and recovering the tegaserod maleate.

42. The process of claim 41, wherein the temperature is of about room temperature.

43. The process of claim 41, wherein the solvent is selected from the group consisting of methanol, ethanol, propanol, xylene, toluene, benzene, propyl-benzene and mixtures thereof.

44. Tegaserod in solid state having a purity of at least about 95% as area percentage HPLC.

45. The tegaserod of claim 44, wherein the purity is at least about 98%.

46. The tegaserod of claim 45, wherein the purity is at least about 99%.

47. Tegaserod having less than about 1% as area percentage HPLC of an impurity characterized by an HPLC RRT of about 1.06 and a molecular weight of 403.

48. The tegaserod of claim 47, wherein the impurity is less than about 0.50 as area percentage HPLC.

49. The tegaserod of claim 48, wherein the impurity is about 0.20 as area percentage HPLC.
